(19) United States
(12) Patent Application Publication
(10) Pub. No.: US 2011/0117614 A1
(43) Pub. Date: May 19, 2011

(54) Production Process for Methionine Using Microorganisms with Reduced Isocitrate Dehydrogenase Activity

(75) Inventors: Oskar Zelder, Speyer (DE); Hartwig Schöder, Nussloch (DE); Corinna Klopprogge, Mannheim (DE); Andrea Herold, Weinheim (DE); Stefan Haefner, Speyer (DE)

(73) Assignee: EVONIK DEGUSSA GMBH, Essen (DE)

(21) Appl. No.: 12/989,772

(22) PCT Filed: Apr. 27, 2009
(86) PCT No.: PCT/EP2009/055051

§ 371 Date: Oct. 26, 2010

(30) Foreign Application Priority Data
Apr. 30, 2008 (DE) 08155428.9

(51) Int. Cl. C12P 13/12

(52) U.S. Cl. 435/113

(57) ABSTRACT

The present invention is directed to a method utilizing a microorganism with reduced isocitrate dehydrogenase activity for the production of methionine.

The present invention is directed to a method utilizing a microorganism with reduced isocitrate dehydrogenase activity for the production of methionine.

### BACKGROUND

Currently worldwide annual production of the amino acid methionine amounts to about 500,000 tons. The standard industrial production process is not by fermentation but a multi-step chemical process. Methionine is the first limiting amino acid in livestock of poultry feed and due to this mainly applied as a feed supplement. Various attempts have been published in the prior art to produce methionine by fermentation e.g. using microorganisms such as E. coli.

Other amino acids such as glutamate, lysine, and threonine, are produced by e.g. fermentation methods. For these purposes, certain microorganisms such as C. glutamicum have been proven to be particularly suited. The production of amino acids by fermentation has the particular advantage that only L-amino acids are produced and that environmentally problematic chemicals such as solvents as they are typically used in chemical synthesis are avoided.

The fermentative production of fine chemicals is today typically carried out in microorganisms such as Corynebacterium glutamicum (C. glutamicum), Escherichia coli (E. coli), Saccharomyces cerevisiae (S. cerevisiae), Schizosaccharomyces pombe (S. pombe), Pichia pastoris (P. pastoris), Aspergillus niger, Bacillus subtilis, Ashbya gossypii or Gluconobacter oxydans. Especially Corynebacterium glutamicum is known for its ability to produce amino acids in large quantities, e.g., L-glutamate and L-lysine (Kinoshita, S. (1985) Glutamic acid bacteria; p. 115-142 in: A. L. Demain and N. A. Solomon (ed.), Biology of industrial microorganisms, Bejamin/cummings Publishing Co., London).

Some of the attempts in the prior art to produce fine chemicals such as amino acids, lipids, vitamins or carbohydrates in microorganisms such as E. coli and C. glutamicum have tried to achieve this goal by e.g. increasing the expression of genes involved in the biosynthetic pathways of the respective fine chemicals. If e.g. a certain step in the biosynthetic pathway of an amino acid such as methionine or lysine is known to be rate-limiting, over-expression of the respective enzyme may allow obtaining a microorganism that yields more product of the catalysed reaction and therefore will ultimately lead to an enhanced production of the respective amino acid. Similarly, if a certain enzymatic step in the biosynthetic pathway of an e.g. desired amino acid is known to be non-desirable as it channels a lot of metabolic energy into formation of undesired by-products it may be contemplated to down-regulate expression of the respective enzymatic activity in order to favour only such metabolic reactions that ultimately lead to the formation of the amino acid in question.

Attempts to increase production of e.g. methionine or lysine by up-and/or downregulating the expression of genes being involved in the biosynthesis of methionine or lysine are e.g. described in WO 02/10209, WO 2006/008097, and WO 2005/059093.

Isocitrate dehydrogenase (ICD, sometimes also called IDH, EC 1.1.1.42, SEQ ID NO:3) is an enzyme which participates in the citric acid cycle (TCA) of, e.g., C. glutamicum (FIG. 1). It catalyzes the third step of the cycle: the oxidative decarboxylation of isocitrate, producing alpha-ketoglutarate and CO₂.

The gene encoding ICD in C. glutamicum was identified, cloned and characterized by Eikmanns et al. (Eikmanns, B. et al., J. Bacteriol. (1995) 177:774-782). Inactivation of the chromosomal icd gene encoding ICD by knockout in C. glutamicum leads to glutamate auxotrophy (Eikmanns, B. et al., J. Bacteriol. (1995) 177:774-782).

Overexpression of ICD in C. glutamicum and E. coli did not enhance glutamate production (Eikmanns, B. et al., J. Bacteriol. (1995) 177:774-782). However, it was reported in DE 10210967 that overexpression of ICD in E. coli leads to an increased threonine production. Contradictory results are reported for the co-expression of icd with the gene encoding glutamate dehydrogenase in C. glutamicum: whilst Eikmanns did not register any effect, an improved glutamate yield is reported in JP63214189 and JP2520895.

Even in view of the reported attempts to increase production of methionine, there is still a need for alternative methods of production.

### OBJECT AND SUMMARY OF THE INVENTION

It is the objective of the present invention to provide alternative fermentative methods and microorganisms for the use in said methods to produce methionine using an industrially important microorganism such as C. glutamicum with heretoforth unknown characteristics.

These and other objectives as they will become apparent from the ensuing description of the invention are solved by the present invention as described in the independent claims. The dependent claims relate to preferred embodiments.

The present invention relates to a method for the production of methionine using cells with a reduced activity of isocitrate dehydrogenase. The downregulation of said enzyme was heretoforth unknown to lead to improved yields of methionine.

The cells used in the production method may be prokaryotes, lower eukaryotes, isolated plant cells, yeast cells, isolated insect cells or isolated mammalian cells, in particular cells in cell culture systems. In the context of present invention, the term “microorganism” is used for said kinds of cells.

A preferred kind of microorganism wherein the ICD activity is reduced for performing the present invention is a Corynebacterium wherein the ICD expression is reduced and particularly preferably a C. glutamicum wherein the ICD expression is reduced.

In particular, the following embodiments of the invention are provided:
(1) a method for the production of methionine, utilizing a microorganism with a partially or completely reduced isocitrate dehydrogenase (ICD) activity in comparison to a corresponding initial microorganism; and
(2) a method of preparing chemicals and chemical end products like polymers from methionine produced by the method according to embodiment (1), comprising as one step the production of said methionine by the method according to embodiment (1).

### FIGURE LEGENDS

FIG. 1: Biochemical pathways in C. glutamicum leading to methionine.

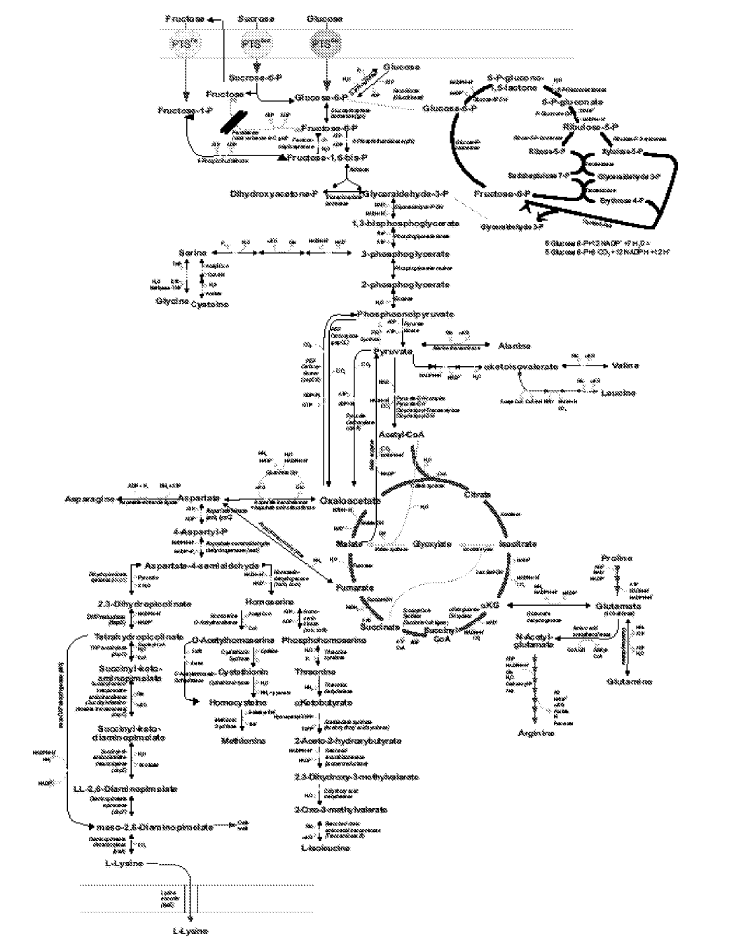

### SEQUENCE LISTING, FREE TEXT

| SEQ ID NO: | Description |
|---|---|
| 1 | wild-type C. glutamicum DNA encoding the ICD of |
| | SED ID NO: 3 |
| 2 | C. glutamicum icd including native DNA sequence 500 |
| | nt up- and downstream of the icd gene |
| 3 | wild-type isocitrate dehydrogenase of C. glutamicum |
| 4 | icd carrying an ATG-GTG mutation (ICD ATG−>GTG) |
| 5 | vector insert that was used to replace the endogenous icd |
| | gene by SEQ ID NO: 4 |
| 6 | codon usage amended isocitrate dehydrogenase (icd) CA2 |
| 7 | vector insert that was used to replace the endogenous icd |
| | gene by SEQ ID NO: 6 |
| 8 | pClik int sacB delta icd |
| 9 | insert of pClik int sacB delta icd |

Definitions

The following abbreviations, terms and definitions are used herein:

IDH, isocitrate dehydrogenase; ICD, isocitrate dehydrogenase; WT, wild type; PPP, pentose phosphate pathway; the abbreviations “ICD” and “IDH” are used synonymously for isocitrate dehydrogenase.

As used in the context of present invention, the singular forms of “a” and “an” also include the respective plurals unless the context clearly dictates otherwise. Thus, the term “a microorganism” can include more than one microorganism, namely two, three, four, five etc. microorganisms of a kind.

The term “about” in context with a numerical value or parameter range denotes an interval of accuracy that the person skilled in the art will understand to still ensure the technical effect of the feature in question. The term typically indicates deviation from the indicated numerical value of +/−10%, preferably +/−5%.

Unless indicated otherwise, a compound or amino acid mentioned in the context of present invention may have any stereochemistry, including a mixture of different steroisomers. Preferably, the amino acids have L-configuration. Specifically preferred configurations are indicated where appropriate.

Unless indicated otherwise, the acids obtained by the method according to present invention may be in the form of a free acid, a partial or complete salt of said acid or in the form of mixtures of the acid and its salt. Vice versa, the amines obtained by the method according to present invention may be in the form of a free amine, a partial or complete salt of said amine or in the form of mixtures of the amine and its salt.

The term “host cell” for the purposes of the present invention refers to any isolated cell that is commonly used for expression of nucleotide sequences for production of e.g. polypeptides or fine chemicals. In particular the term “host cell” relates to prokaryotes, lower eukaryotes, plant cells, yeast cells, insect cells or mammalian cell culture systems.

The term “microorganism” relates to prokaryotes, lower eukaryotes, isolated plant cells, yeast cells, isolated insect cells or isolated mammalian cells, in particular cells in cell culture systems. The microorganisms suitable for performing the present invention comprise yeasts such as S. pombe or S. cerevisiae and Pichia pastoris. Mammalian cell culture systems may be selected from the group comprising e.g. NIH T3 cells, CHO cells, COS cells, 293 cells, Jurkat cells and HeLa cells. In the context of present invention, a microorganism is preferably a prokaryote or a yeast cell. Preferred microorganisms in the context of present invention are indicated below in the “detailed description” section. Particularly preferred are Corynebacteria.

“Native” is a synonym for “wild type” and “naturally occurring”. A “wild-type” microorganism is, unless indicated otherwise, the common naturally occurring form of the indicated microorganism. Generally, a wild-type microorganism is a non-recombinant microorganism.

“Initial” is a synonym to “starting”. An “initial” nucleotide sequence or enzyme activity is the starting point for its modification, e.g. by mutation or addition of inhibitors. Any “initial” sequence, enzyme or microorganism lacks a distinctive feature which its “final” or “modified” counterpart possesses and which is indicated in the specific context (e.g. a reduced ICD activity). The term “initial” in the context of present invention encompasses the meaning of the term “native”, and in a preferred aspect is a synonym for “native”.

Any wild-type or mutant (non-recombinant or recombinant mutant) microorganism may be further modified by non-recombinant (e.g. addition of specific enzyme inhibitors) or recombinant methods resulting in a microorganism which differs for the initial microorganism in at least one physical or chemical property, and in one particular aspect of present invention in its ICD activity. In the context of present invention, the initial, non-modified microorganism is designated as “initial microorganism” or “initial (microorganism) strain”. Any reduction of ICD activity in a microorganism in comparison to the initial strain with a given ICD expression level is determined by comparison of ICD activity in both microorganisms under comparable conditions.

Typically, microorganisms in accordance with the invention are obtained by introducing genetic alterations in an intial microorganism which does not carry said genetic alteration.

A “derivative” of a microorganism strain is a strain that is derived from its parent strain by e.g. classical mutagenesis and selection or by directed mutagenesis. E.g., the strain C. glutamicum ATCC13032lysC^{fbr} (WO 2005/059093) is a lysine production strain derived from ATCC13032.

The term “nucleotide sequence” or “Nucleic acid sequence” for the purposes of the present invention relates to any nucleic acid molecule that encodes for polypeptides such as peptides, proteins etc. These nucleic acid molecules may be made of DNA, RNA or analogues thereof. However, nucleic acid molecules made of DNA are preferred.

“Recombinant” in the context of present invention means “being prepared by or the result of genetic engineering”. Thus, a “recombinant microorganism” comprises at least one “recombinant nucleic acid” or “recombinant protein”. A recombinant microorganism preferably comprises an expression vector or cloning vector, or it has been genetically engineered to contain the cloned nucleic acid sequence(s) in the endogenous genome of the host cell.

“Heterologous” is any nucleic acid or polypeptide/protein introduced into a cell or organism by genetic engineering with respect to said cell or organism, and irrespectively of its organism of origin. Thus, a DNA isolated from a microorganism and introduced into another microorganism of the same species is a heterologous DNA with respect to the latter, genetically modified microorganism in the context of present invention, even though the term “homologous” is sometimes used in the art for this kind of genetically engineered modifications. However, the term “heterologous” is preferably addressing a non-homologous nucleic acid or polypeptide/protein in the context of present invention. “Heterologous protein/nucleic acid” is synonymous to “recombinant protein/nucleic acid”.

The terms “express”, “expressing,” “expressed” and “expression” refer to expression of a gene product (e.g., a biosynthetic enzyme of a gene of a pathway) in a host organism. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

A “conservative amino acid exchange” means that one or more amino acids in an initial amino acid sequence are substituted by amino acids with similar chemical properties, e.g. Val by Ala. The ratio of substituted amino acids in comparison to the initial polypeptide sequence is preferably from 0 to 30% of the total amino acids of the initial amino acid sequence, more preferably from 0 to 15%, most preferably from 0 to 5%.

Conservative amino acid exchanges are preferably between the members of one of the following amino acid groups:
acidic amino acids (aspartic and glutamic acid);
basic amino acids (lysine, arginine, histidine);
hydrophobic amino acids (leucine, isoleucine, methionine, valine, alanine);
hydrophilic amino acids (serine, glycine, alanine, threonine);
amino acids having aliphatic side chains (glycine, alanine, valine, leucine, isoleucine);
amino acids having aliphatic-hydroxyl side chains (serine, threonine);
amino acids having amide-containing side chains (asparagine, glutamine);
amino acids having aromatic side chains (phenylalanine, tyrosine, tryptophan);
amino acids having basic side chains (lysine, arginine, histidine);
amino acids having sulfur-containing side chains (cysteine, methionine).

Specifically preferred conservative amino acid exchanges are as follows:

| Native residue | Substituting residue |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term “isolated” means “separate or purified from its organism of origin”. More specifically, an isolated cell of a multicellular organism is separate or has been purified from its organism of origin. This encompasses biochemically purified and recombinantly produced cells.

As used herein, a “precursor” or “biochemical precursor” of an amino acid is a compound preceding (“upstream”) the amino acid in the biochemical pathway leading to the formation of said amino acid in the microorganism of present invention, especially a compound formed in the last few steps of said biochemical pathway. In the context of present invention, a “precursor” of methionine is any intermediate formed during biochemical conversion of aspartate to methionine in a wild-type organism in vivo.

“Carbon yield” is the carbon amount found (of the product) per carbon amount consumed (of the carbon source used in the fermentation, usually a sugar), i.e. the carbon ratio of product to source.

“ICD activity” in the context of present invention means any enzymatic activity of ICD, especially any catalytic effect exerted by ICD. Specifically, the conversion of isocitrate into alpha-ketoglutarate is meant by “ICD activity”. ICD activity may be expressed as units per milligram of enzyme (specific activity) or as molecules of substrate transformed per minute per molecule of enzyme.

### DETAILED DESCRIPTION OF THE INVENTION

The present invention pertains to the biochemical synthesis of methionine by a microorganism with reduced ICD activity.

The activity of ICD provides some of the NADPH/NADH necessary for the amino acid production in a cell. Thus, it did not seem obvious previous to the conception of present invention to reduce ICD activity in a cell in order to amplify its methionine production.

Surprisingly, it was now found that a reduction of the ICD activity in a microorganism leads to an increased level of production of methionine. Methionine is of considerable interest as fine chemical.

In a preferred aspect of present invention, the production method according to embodiment (1) is a fermentative method. However, other methods of biotechnological production of chemical compounds are also considered, including in vivo production in plants and non human animals.

The method for the fermentative production of methionine according to embodiment (1) may comprise the cultivation of at least one—preferably recombinant—microorganism having a reduced ICD activity such that the carbon flux through the glyoxylate shunt is increased.

In a further preferred aspect of embodiment (1), the microorganism used in the production method is a recombinant microorganism. Inasfar as other methods of biotechnological production of chemical compounds are also considered, including in vivo production in plants and non human animals, the organism of choice is preferably a recombinant organism.

In any embodiment of present invention, the isocitrate dehydrogenase activity in the microorganism used for the embodiment is partially or completely reduced.

A microorganism having a reduced ICD activity according to present invention has lost its native ICD activity partially or completely when compared with an initial microorganism of the same species and genetical background. Preferably, about at least 1%, at least 2%, at least 4%, at least 6%, at least 8%, at least 10%, more preferably at least 20%, at least 40%, at least 60%, at least 80%, at least 90%, at least 95% or all of the initial activity of ICD is lost in the microorganism. The extent of reduction of activity is determined in comparison to the level of activity of the endogenous ICD activity in an intial microorganism under comparable conditions.

It is understood that it is not always desirable to reduce ICD activity as much as possible. In certain cases an incomplete reduction of any of the levels indicated above, but also of intermediate levels like, e.g., 25%, 40%, 50% etc., may be sufficient and desirable.

An incomplete loss of ICD activity is preferred, as this keeps up the TCA and allows the microorganism to further produce glutamate and other biomolecules synthesized from alpha-ketoglutarate.

In embodiments wherein a complete or near complete (i.e. 90% or greater) loss of ICD activity characterizes the microorganism, the cultivation media for the microorganism, especially the media used in the production according to embodiment (1) may be supplemented by one or more essential compounds lacking in the microorganism due to the suppression of ICD activity. Especially glutamate may be supplemented th the media as it is an inexpensive, easily accessable compound.

In organisms possessing more than one ICD encoding gene and/or more than one kind of ICD, the ICD activity reduction may be a reduction in activity of all, several or only one of the different kinds of ICD. A specific reduction of less than all kinds of ICD is preferred for the reasons indicated above in context with the incomplete loss of ICD.

The reduction of ICD activity necessary for present invention may be either an endogenous trait of the microorganism used in the method according to embodiment (1), e.g. a trait due to spontaneous mutations, or due to any method known in the art for suppressing or inhibiting an enzymatic activity in part or completely, especially an enzymatic activity in vivo. The reduction of enzymatic activity may occur at any stage of enzyme synthesis and enzyme reactions, at the genetic, transcription, translation or reaction level.

The decrease of ICD activity is preferably the result of genetic engineering. To reduce the amount of expression of one or more endogenous ICD gene(s) in a host cell and to thereby decrease the amount and/or activity of the ICD in the host cell in which the icd target gene is suppressed, any method known in the art may be applied. For down-regulating expression of a gene within a microorganism such as E. coli or C. glutamicum or other host cells such as P. pastoris and A. niger, a multitude of technologies such as gene knockout approaches, antisense technology, RNAi technology etc. are available. One may delete the initial copy of the respective gene and/or replace it with a mutant version showing decreased activity, particularly decreased specific activity, or express it from a weak promoter. Or one may exchange the promoter of an icd gene, introduce mutations by random or target mutagenesis, disrupt or knock-out an icd gene. Furtheron, one may introduce destabilizing elements into the mRNA or introduce genetic modifications leading to deterioration of ribosomal binding sites (RBS) of the RNA. Finally, one may add specific ICD inhibitors to the reaction mixture.

In a first preferred aspect of embodiment (1), the ICD activity is reduced due to partial or complete reduction of ICD expression. “Reducing the expression of at least one ICD in a microorganism” refers to any reduction of expression in a microorganism in comparison to an initial microorganism with a given ICD expression level. This, of course, assumes that the comparison is made for comparable host cell types, comparable genetic background situations etc. Preferably, the reduction of expression is achieved as listed above or described in the following.

In a particular aspect of present invention, the microorganism has lost its initial ICD activity due to a decrease in ICD expression, preferably a decrease by at least about 5%, 10%, 20%, 30%, 40%, 50%, 60%, 70%, 80%, 90%, 95% or 100%, with the extent of reduction of expression being determined in comparison to the level of expression of the polypeptide in an initial microorganism. The extent of reduction of expression is determined in comparison to the level of expression of the endogenous ICD that is expressed from the initial icd nucleotide sequence in an intial microorganism under comparable conditions.

In organisms possessing more than one ICD encoding gene and/or more than one kind of ICD, the reduction of ICD expression may concern one, several or all icd genes. A specific reduction of expression of less than all icd genes is preferred for the reasons indicated above in context with the incomplete loss of ICD.

In one preferred aspect, “reduction of expression” means the situation that if one replaces an endogenous nucleotide sequence coding for a polypeptide with a modified nucleotide sequence that encodes for a polypeptide of substantially the same amino acid sequence and/or function, a reduced amount of the encoded polypeptide will be expressed within the modified cells.

A specific aspect of this downregulation mode is the knock-out of the icd gene (compare example 3). It may be achieved by any known knock-out protocol suitable for the microorganism in question. Particularly preferred methods for knock-out and for production of methionine using the resulting knock-out mutants are described in example 2.

The knock-out of the icd may lead to complete or near-complete loss of ICD activity. Thus, in order to avoid deficiency symptoms and to keep the microorganism alive, a supplementation of the culturing media with deficient ICD-dependent products like glutamate may be necessary for knock-out mutants.

In a further preferred aspect, “reduction of expression” means the down-regulation of expression by antisense technology or RNA interference (where applicable, e.g. in eucaryotic cell cultures) to interfere with gene expression. These techniques may affect icd mRNA levels and/or icd translational efficiency.

In yet a further preferred aspect, “reduction of expression” means the deletion or disruption of the icd gene combined with the introduction of a “weak” icd gene, i.e. a gene encoding an ICD whose enzymatic activity is lower than the initial ICD activity, or by integration of the icd site at a weakly expressed site resulting in less ICD activity inside the cell. This may be done by integrating the icd gene at a chromosomal locus from which genes are less well transcribed, or by introducing a mutant or heterologous icd gene with lower specific activity or which is less efficiently transcribed, less efficiently translated or less stable in the cell. The introduction of this mutant icd gene can be performed by using a replicating plasmid or by integration into the genome.

In yet a further preferred aspect, “reduction of expression” means that the reduced ICD activity is the result of lowering the mRNA levels by lowering transcription from the chromosomally encoded icd gene, preferably by mutation of the initial promoter or replacement of the native ICD promoter by a weakened version of said promoter or by a weaker heterologous promoter. Particularly preferred methods for performing this aspect and for production of methionine using the resulting mutants are described in example 4.

In yet a further preferred aspect, “reduction of expression” means that the reduced ICD activity is the result of RBS mutation leading to a decreased binding of ribosomes to the translation initiation site and thus to a decreased translation of icd mRNA. The mutation can either be a simple nucleotide change and/or also affect the spacing of the RBS in relation to the start codon. To achieve these mutations, a mutant library containing a set of mutated RBSs may be generated. A suitable RBS may be selected, e.g. by selecting for lower ICD activity. The initial RBS may then be replaced by the selected RBS. Particularly preferred methods for performing this aspect and for production of methionine using the resulting mutants are described in example 4.

In yet a further preferred aspect, “reduction of expression” is achieved by lowering mRNA levels by decreasing the stability of the mRNA, e.g. by changing the secondary structure.

In yet a further preferred aspect, “reduction of expression” is achieved by icd regulators, e.g. transcriptional regulators.

A specific method for dowregulating ICD expression in yet a further preferred aspect is the codon usage method described in PCT/EP2007/061151, which is hereby incorporated by reference inasfar as application of the codon usage method for downregulating ICD activity in microorganisms, especially in Corynebacterium and E. coli is concerned.

PCT/EP2007/061151 describes a method of reducing the amount of at least one polypeptide in a host cell, comprising the step of expressing in said host cell a modified nucleotide sequence instead of a non-modified nucleotide sequence encoding for a polypeptide of substantially the same amino acid sequence and/or function wherein said modified nucleotide sequence is derived from the non-modified nucleotide sequence such that at least one codon of the non-modified nucleotide sequence is replaced in the modified nucleotide sequence by a less frequently used codon according to the codon usage of the host cell.

In case of modified nucleotide sequences that are to be expressed in Corynebacterium and particularly preferably in C. glutamicum for reducing the amount of the ICD, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequences may be replaced in the modified nucleotide sequence by less frequently used codons for the respective amino acid. In an even more preferred embodiment the afore-mentioned number of codons to be replaced refers to frequent, very frequent, extremely frequent or the most frequent codons. In another particularly preferred embodiment, the above number of codons are replaced by the least frequently used codons. In all these cases will the reference codon usage be based on the codon usage of the Corynebacterium and preferably C. glutamicum and preferably on the codon usage of abundant proteins of Corynebacterium and preferably C. glutamicum. See also PCT/EP2007/061151 for detailed explanation.

A particularly preferred aspect of the invention relates to a method wherein the decrease of the expression of isocitrate dehydrogenase in a microorganism is achieved by adapting the codon usage as described in PCT/EP2007/061151. The microorganism can be a Corynebacterium, with C. glutamicum being preferred. These methods may be used to improve synthesis of methionine. Thus, microorganisms with a reduced ICD activity due to application of the codon usage method described in PCT/EP2007/061151 are in one preferred aspect of present invention the microorganisms of choice for performing the method according to embodiment (1). PCT/EP2007/061151 does especially describe the reduction of ICD in C. glutamicum cells by replacement of the start codon with GTG in one embodiment and by change of a glycine and an isoleucine codon from GGC ATT to GGG ATA at positions 32 and 33 of native ICD (compare example 1). These two embodiments of PCT/EP2007/061151 are the microorganisms of choice in one aspect of the production method of embodiment (1) and their use in the method according to embodiment (1) of present invention is therefore specifically incorporated by reference. Their preparation and use is demo strated in example 1.

On the other hand, in a different particularly preferred aspect of present invention, microorganisms with a reduced ICD activity due to application of the codon usage method described in PCT/EP2007/061151 are excluded from being the microorganisms of choice in the method according to embodiment (1). According to said aspect, the method of embodiment (1) is an embodiment of present invention with the proviso that the reduction of ICD expression is not due to the expression of a modified ICD encoding nucleotide sequence (icd sequence) instead of the native icd sequence of the microorganism wherein said modified icd encoding sequence is derived from the non-modified icd sequence such that at least one codon of the non-modified nucleotide sequence is replaced in the modified icd sequence by a less frequently used codon according to the codon usage of the host cell. In other words, the method of embodiment (1) is an embodiment of present invention with the proviso that the reduction of ICD expression is not due to modified codon usage as described in PCT/EP2007/061151 and that no microorganism described in PCT/EP2007/061151 is used. More preferably, the method of embodiment (1) is an embodiment of present invention with the proviso that, when methionine is produced, the reduction of ICD expression is not due to the expression of a modified ICD encoding nucleotide sequence (icd sequence) instead of the native icd sequence of the microorganism wherein said modified icd encoding sequence is derived from the non-modified icd sequence such that at least one codon of the non-modified nucleotide sequence is replaced in the modified icd sequence by a less frequently used codon according to the codon usage of the microorganism.

In a second preferred aspect of embodiment (1), the ICD activity is reduced due to partial or complete inhibition of the enzyme. The inhibition may be the result of binding of any known reversible or irreversible ICD inhibitor to ICD. Such inhibitors are known in the art, e.g. oxaloacetate, 2-oxoglutarate and citrate which are known as weak inhibitors of ICD in C. glutamicum, or oxaloacetate and glyoxylate, which are known as strong inhibitors (Eikmanns et al (1995) loc. cit.). Said inhibitor may either be added to the fermentation medium, or its synthesis inside the cell may be induced by an external stimulus.

In several preferred aspects of embodiment (1) and (2), the reduced ICD activity is the result of genetically engineering a host cell (preferably a microorganism, especially a Corynebacterium), but not the result of reduced ICD expression.

Particularly, in a third preferred aspect, deleting the initial copy of an icd gene and replacing it with a mutant version encoding an ICD that shows decreased ICD activity or with a heterologous icd gene encoding an ICD having less ICD activity than the initial ICD, leads to a decrease in ICD activity of the microorganism of present invention. Particularly preferred methods for performing this aspect and for production of methionine using the resulting mutants are described in example 3.

In a fourth preferred aspect, a combination of two or more of the aforementioned features leading to ICD activity reduction is realized in the microorganism according present invention.

A preferred method in accordance with embodiment (1) of the present invention comprises the step of reducing the ICD acitivity in a microorganism, preferably in Corynebacteria and more preferably in C. glutamicum, wherein the above principles are used.

The increase in biosysnthesis of methionine in a microorganism with reduced ICD activity may be due to an increased carbon flux through PPP and glyoxylate shunt as a result of ICD inhibition. The former leads to provision of sufficient reduction equivalents, i.e. NAD(P)H, for amino acid production, the latter provides the necessary carbon precursors for biosynthesis of methionine. Thus, in one preferred aspect of present invention, in the microorganism used in embodiment (1) or the microorganism according to embodiment (2), the carbon flux through

(i) the glyoxylate shunt and/or

(ii) the pentose phosphate pathway (PPP)

is increased in comparison to a wild-type microorganism. Preferably, the carbon flux through the glyoxylate shunt is increased. Any of said increases may be the result of the ICD activity reduction, the result of genetically engineering the microorganism, a native trait of the microorganism, or a combination of any of these factors. The increased carbon flux through the glyoxylate shunt is preferably the result of the ICD activity reduction and/or of genetically engineering the microorganism. The increased carbon flux through PPP is preferably the result of genetically engineering the microorganism, more preferably the result of an active upregulation of the PPP enzyme expression level, e.g. by using a strong promoter like Psod (WO 2005/059144).

As indicated above, the present invention pertains to microorganisms and to the use of microorganisms in methionine production. However, the use of other organism besides microorganisms in the production method according to embodiment (1) is also contemplated. The term “organism” for the purposes of the present invention refers to any non-human organism that is commonly used for expression of nucleotide sequences for production of fine chemicals, in particular microorganisms as defined above, plants including algae and mosses, yeasts, and non-human animals. Organisms besides microorganisms which are particularly suitable for fine chemical production are plants and plant parts. Such plants may be monocots or dicots such as monocotyledonous or dicotyledonous crop plants, food plants or forage plants. Examples for monocotyledonous plants are plants belonging to the genera of avena (oats), triticum (wheat), secale (rye), hordeum (barley), oryza (rice), panicum, pennisetum, setaria, sorghum (millet), zea (maize) and the like.

Dicotyledonous crop plants comprise inter alia cotton, leguminoses like pulse and in particular alfalfa, soybean, rapeseed, tomato, sugar beet, potato, ornamental plants as well as trees. Further crop plants can comprise fruits (in particular apples, pears, cherries, grapes, citrus, pineapple and bananas), oil palms, tea bushes, cacao trees and coffee trees, tobacco, sisal as well as, concerning medicinal plants, rauwolfia and digitalis. Particularly preferred are the grains wheat, rye, oats, barley, rice, maize and millet, sugar beet, rapeseed, soy, tomato, potato and tobacco. Further crop plants can be taken from U.S. Pat. No. 6,137,030.

The person skilled in the art is well aware that different organisms and cells such as microorganisms, plants and plant cells, animals and animal cells etc. will differ with respect to the number and kind of icd genes and ICD proteins in a cell. Even within the same organism, different strains may show a somewhat heterogeneous expression profile on the protein level.

In case an organism different from a microorganism is used in performing the present invention, a non-fermentative production method may be applied.

In present invention according to embodiments (1) and (2), any microorganism as defined above may be used. Preferably, the microorganism is a prokaryote. Particularly preferred for performing the present invention are microorganisms being selected from the genus of Corynebacterium and Brevibacterium, preferably Corynebacterium, with a particular focus on Corynebacterium glutamicum, the genus of Escherichia with a particular focus on Escherichia coli, the genus of Bacillus, particularly Bacillus subtilis, the genus of Streptomyces and the genus of Aspergillus.

A preferred embodiment of the invention relates to the use of microorganisms which are selected from coryneform bacteria such as bacteria of the genus Corynebacterium. Particularly preferred are the species Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola and Corynebacterium effiziens. Other preferred embodiments of the invention relate to the use of Brevibacteria and particularly the species Brevibacterium flavum, Brevibacterium lactofermentum and Brevibacterium divarecatum.

In preferred embodiments of the invention the microorganism may be selected from the group consisting of Corynebacterium glutamicum ATCC13032, C. acetoglutamicum ATCC15806, C. acetoacidophilum ATCC13870, Corynebacterium thermoaminogenes FERMBP-1539, Corynebacterium melassecola ATCC17965, Corynebacterium effiziens DSM 44547, Corynebacterium effiziens DSM 44549, Brevibacterium flavum ATCC14067, Brevibacterium lactoformentum ATCC13869, Brevibacterium divarecatum ATCC 14020, Corynebacterium glutamicum KFCC10065 and Corynebacterium glutamicum ATCC21608 as well as strains that are derived thereof by e.g. classical mutagenesis and selection or by directed mutagenesis.

Other preferred strains of C. glutamicum may be selected from the group consisting of ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12417, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen and Zellkulturen. The abbreviation NRRL stands for ARS cultures collection Northern Regional Research Laboratory, Peorea, Ill., USA.

Strains of Corynebacterium glutamicum that are already capable of producing fine chemicals such as L-lysine, L-methionine, L-isoleucine and/or L-threonine are particularly preferred for performing present invention. Such a strain is e.g. Corynebacterium glutamicum ATCC13032 and derivatives thereof. The strains ATCC 13286, ATCC 13287, ATCC 21086, ATCC 21127, ATCC 21128, ATCC 21129, ATCC 21253, ATCC 21299, ATCC 21300, ATCC 21474, ATCC 21475, ATCC 21488, ATCC 21492, ATCC 21513, ATCC 21514, ATCC 21515, ATCC 21516, ATCC 21517, ATCC 21518, ATCC 21528, ATCC 21543, ATCC 21544, ATCC 21649, ATCC 21650, ATCC 21792, ATCC 21793, ATCC 21798, ATCC 21799, ATCC 21800, ATCC 21801, ATCC 700239, ATCC 21529, ATCC 21527, ATCC 31269 and ATCC 21526 which are known to produce lysine can also preferably be used. Particularly preferred are Corynebacterium glutamicum strains that are already capable of producing fine chemicals such as L-lysine, L-methionine and/or L-threonine. Therefore the strain Corynebacterium glutamicum ATCC13032 and derivatives of this strain are particularly preferred. This preference encompasses the strains ATCC13032lysC^{fbr}, and ATCC13286. C. glutamicum ATCC13032lysC^{fbr}, ATCC13032 or ATCC13286 are specifically preferred microorganisms in the context of present invention.

It is understood that in order to be suitable for present invention all the microorganisms listed above will display a partially or completely reduced ICD activity. Preferred microorganisms in the context of present invention are recombinant microorganisms whose reduced ICD activity is the result of genetic engineering.

Embodiment (1) of present invention concerns the use of an aforementioned microorganism having a reduced ICD activity to produce methionine, especially L-methionine.

Methionine can be used in different parts of the pharmaceutical industry, agricultural industry as well as in the cosmetics, food and feed industry.

For the method according to embodiment (1), a microorganism may be used which does not only possess reduced ICD activity, but is also specifically adapted for production of methionine. This adaptation may be due to a repression or reduction of enzyme activities known to be responsible for the synthesis of unwanted by-products/side products. Lowering the amount or activity of an enzyme that forms part of a biosynthetic pathway may allow increasing synthesis of methionine by e.g. shutting off production of by-products and by channelling metabolic flux into the methionine biosynthetic pathway.

On the other hand, this adaptation may be due to an increased activity of enzymes in methionine biosynthesis. It is preferred that said adaption of the microorganism encompasses an increase of activity and/or expression of an enzyme which catalyzes one or more than one of the conversion steps leading up to methionine, in particular of an enzyme catalyzing a conversion step downstream of aspartate, more particularly of an enzyme catalysing a conversion step in the conversion of aspartate to methionine. It is further preferred that said adaptation is due to genetic engineering leading to the presence of at least one heterologous enzyme in the microorganism which enhances the production of methionine.

In a preferred embodiment of the method (1) of present invention, one or more than one further enzyme activity besides the ICD activity in endogenous biosynthetic pathways of the miccroorganism is modified, leading to an increase of carbon yield for the target compound methionine. Preferably, one or more than one of the enzymes catalyzing the biochemical transformation of aspartate to lysine, methionine or isoleucine is up- or downregulated.

Preferably, the activity of a Corynebacterium enzyme and particularly of a C. glutamicum enzyme is up- or downregulated.

Preferably, said modification is achieved by modification of the nucleotide sequences encoding said enzymes.

Modified enzymes and/or nucleotide sequences which are preferably down-regulated may be selected from the group consisting of sequences encoding homoserine-kinase, threonine-dehydratase, threonine-synthase, meso-diaminopimelat D-dehydrogenase, phosphoenolpyruvate-carboxykinase, pyruvat-oxidase, dihydrodipicolinate-synthase, dihydrodipicolinate-reductase, and diaminopicolinate-decarboxylase. Preferably, said enzymes are downregulated. Of these, theo following are preferred for down-regulation: homoserine-kinase, phosphoenolpyruvate-carboxykinase and dihydrodipicolinate-synthase.

The gene products which are preferably upregulated are selected from the following group: Cystathionin Synthase, Cystathionin lyase, homoserine-O-acetyltransferase, O-acetylhomoserine-sulfhydrylase, homoserine-dehydrogenase, aspartate-kinase, aspartate-semialdehyde-dehydrogenase, glycerinaldehyde-3-phosphate-dehydrogenase, 3-phosphoglycerate-kinase, pyruvate-carboxylase, triosephosphate-isomerase, transaldolase, transketolase, glucose-6-phosphate-dehydrogenase, biotine-ligase, protein OpcA, 1-phosphofructo-kinase, 6-phosphofructo-kinase, fructose-1,6-bisphosphatase, 6-phosphogluconate-dehydrogenase, homoserine-dehydrogenase, phosphoglycerate-mutase, pyruvat-kinase, aspartate-transaminase, coenzym B12-dependent methionine-synthase, coenzym B12-independent methione-synthase and malate-enzyme.

Embodiment (1) may further include a step of recovering the target compound methionine. The term “recovering” includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like. For example the target compound can be recovered from culture media by first removing the microorganisms. The remaining broth is then passed through or over a cation exchange resin to remove unwanted cations and then through or over an anion exchange resin to remove unwanted inorganic anions and organic acids.

In embodiment (2) the present invention provides a method for the production of further products made from the methionine prepared by the method according to embodiment (1). A person skilled in the art is familiar with how to replace e.g. a gene or endogenous nucleotide sequence that encodes for a certain polypeptide with a modified nucleotide sequence. This may e.g. be achieved by introduction of a suitable construct (plasmid without origin of replication, linear DNA fragment without origin of replication) by electroporation, chemical transformation, conjugation or other suitable transformation methods. This is followed by e.g. homologous recombination using selectable markers which ensure that only such cells are identified that carry the modified nucleotide sequence instead of the endogenous naturally occurring sequence. Other methods include gene disruption of the endogenous chromosomal locus and expression of the modified sequences from e.g. plasmids. Yet other methods include e.g. transposition. Further information as to vectors and host cells that may be used will be given below.

In general, the person skilled in the art is familiar with designing constructs such as vectors for driving expression of a polypeptide in microorganisms such as E. coli and C. glutamicum. The person skilled in the art is also well acquainted with culture conditions of microorganisms such as C. glutamicum and E. coli as well as with procedures for harvesting and purifying methionine from the aforementioned microorganisms. Some of these aspects will be set out in further detail below.

The person skilled in the art is also well familiar with techniques that allow to change the original non-modified nucleotide sequence into a modified nucleotide sequence encoding for polypeptides of identical amino acid but with different nucleic acid sequence. This may e.g. be achieved by polymerase chain reaction based mutagenesis techniques, by commonly known cloning procedures, by chemical synthesis etc. Standard techniques of recombinant DNA technology and molecular biology are described in various publications, e.g. Sambrook et al. (2001), Molecular Cloning: A Laboratory Manual, 3rd edition, Cold Spring Harbor Laboratory Press, or Ausubel et al. (eds) Current protocols in molecular biology. (John Wiley & Sons, Inc. 2007). Ausubel et al., Current Protocols in Protein Science, (John Wiley & Sons, Inc. 2002). Ausubel et al. (eds.), SHORT PROTOCOLS IN MOLECULAR BIOLOGY, 3rd Edition (John Wiley & Sons, Inc. 1995). Methods specifically for C. glutamicum are described in Eggeling and Bott (eds) Handbook of Corynebacterium (Taylor and Francis Group, 2005). Some of these procedures are set out below and in the “examples” section.

In the following, it will be described and set out in detail how genetic manipulations in microorgansims such as E. coli and particularly Corynebacterium glutamicum can be performed.

Vectors and Host Cells

As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e. g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e. g., replication defective retroviruses, adenoviruses and adeno-associated viruses), which serve equivalent functions.

A recombinant expression vector suitable for preparation of the recombinant microorganism of the invention may comprise a heterologous nucleic acid as defined above in a form suitable for expression of the respective nucleic acid in a host cell, which means that the recombinant expression vectors include one or more regulatory sequences, selected on the basis of the host cells to be used for expression, which is operatively linked to the nucleic acid sequence to be expressed.

Within a recombinant expression vector, “operably linked” is intended to mean that the nucleotide sequence of interest is linked to the regulatory sequence (s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-, tet-, lpp-, lac-, lpp-lac-, lacIq-, T7-, T5-, T3-, gal-, trc-, ara-, SP6-, arny, SP02, e-Pp-orc PL, SOD, EFTu, EFTs, GroEL, MetZ (last 5 from C. glutamicum), which are used preferably in bacteria. Additional regulatory sequences are, for example, promoters from yeasts and fungi, such as ADC1, MFa, AC, P-60, CYC1, GAPDH, TEF, rp28, ADH, promoters from plants such as CaMV/355, SSU, OCS, lib4, usp, STLS1, B33, nos or ubiquitin-or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors can be introduced into host cells to thereby produce proteins or peptides, including fusion proteins or peptides.

Any vector that is suitable to drive expression of a modified nucleotide sequence in a host cell, preferably in Corynebacterium and particularly preferably in C. glutamicum may be used for decreasing the amount of ICD in these host cells. Such vector may e.g. be a plasmid vector which is autonomously replicable in coryneform bacteria. Examples are pZ1 (Merkel et al. (1989), Applied and Environmental Microbiology 64: 549-554), pEKEx1 (Eikmanns et al. (1991), Gene 102: 93-98), pHS2-1 (Sonnen et al. (1991), Gene 107: 69-74) These vectors are based on the cryptic plasmids pHM1519, pBL1 oder pGA1. Other suitable vectors are pCLiK5MCS (WO2005059093), or vectors based on pCG4 (U.S. Pat. No. 4,489,160) or pNG2 (Serwold-Davis et al. (1990), FEMS Microbiology Letters 66, 119-124) or pAG1 (U.S. Pat. No. 5,158,891). Examples for other suitable vectors can be found in the Handbook of Corynebacterium, Chapter 23 (edited by Eggeling and Bott, ISBN 0-8493-1821-1, 2005).

Recombinant expression vectors can be designed for expression of specific nucleotide sequences in prokaryotic or eukaryotic cells. For example, the nucleotide sequences can be expressed in bacterial cells such as C. glutamicum and E. coli, insect cells (using baculovirus expression vectors), yeast and other fungal cells (see Romanos, M. A. et al. (1992), Yeast 8: 423-488; van den Hondel, C. A. M. J. J. et al.(1991) in: More Gene Manipulations in Fungi, J. W. Bennet & L. L. Lasure, eds., p. 396-428: Academic Press: San Diego; and van den Hondel, C. A. M. J. J. & Punt, P. J. (1991) in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) Plant Cell Rep.: 583-586). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve four purposes: 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion E. coli expression vectors include pTrc (Amann et al., (1988) Gene 69: 301-315), pLG338, pACYC184, pBR322,pUC18, pUC19, pKC30, pRep4,pHS1, pHS2, pPLc236, pMBL24, pLG200, pUR290,pIN-III113-B1, egtll, pBdCl, an pET lld (Studier etal., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET lld vector relies on transcription from a T7 gnlO-lac fusion promoter mediated by a coexpressed viral RNA polymerase (T7gnl). This viral polymerase is supplied by host strains BL21 (DE3) or HMS174 (DE3) from a resident X prophage harboring a T7gnl gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194 or pBD214 are suited for transformation of Bacillus species. Several plasmids of use in the transfer of genetic information into Corynebacterium include pHM1519, pBL1, pSA77 or pAJ667 (Pouwels et al., eds. (1985) Cloning Vectors. Elsevier: New York IBSN 0 444 904018).

Examples of suitable C. glutamicum and E. coli shuttle vectors are e.g. pClik5aMCS (WO 2005/059093) or can be found in Eikmanns et al (Gene. (1991) 102, 93-8).

Examples for suitable vectors to manipulate Corynebacteria can be found in the Handbook of Corynebacterium (edited by Eggeling and Bott, ISBN 0-8493-1821-1, 2005). One can find a list of E. coli-C. glutamicum shuttle vectors (table 23.1), a list of E. coli-C. glutamicum shuttle expression vectors (table 23.2), a list of vectors which can be used for the integration of DNA into the C. glutamicum chromosome (table 23.3), a list of expression vectors for integration into the C. glutamicum chromosome (table 23.4.) as well as a list of vectors for site-specific integration into the C. glutamicum chromosome (table 23.6).

In another embodiment, the expression vector is a yeast expression vector. Examples of vectors for expression in yeast S. cerevisiae include pYepSecl (Baldari, et al., (1987) Embo J. 6: 229-234), 2i, pAG-1, Yep6, Yep13, pEMBLYe23, pMFa (Kurjan and Herskowitz, (1982) Cell 30: 933-943), pJRY88 (Schultz et al., (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

For the purposes of the present invention, an operative link is understood to be the sequential arrangement of promoter (including the ribosomal bindung site (RBS)), coding sequence, terminator and, optionally, further regulatory elements in such a way that each of the regulatory elements can fulfill its function, according to its determination, when expressing the coding sequence.

In another embodiment, heterologous nucleotide sequences may be expressed in unicellular plant cells (such as algae) or in plant cells from higher plants (e. g., the spermatophytes, such as crop plants). Examples of plant expression vectors include those detailed in: Becker, D., Kemper, E., Schell, J. and Masterson, R. (1992) Plant Mol. Biol. 20: 1195-1197; and Bevan, M. W. (1984) Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

In another embodiment, a recombinant mammalian expression vector is capable of directing expression of a nucleic acid preferentially in a particular cell type, e.g. in plant cells (e. g., tissue-specific regulatory elements are used to express the nucleic acid). Tissue-specific regulatory elements are known in the art.

Another aspect of the invention pertains to the use of organisms or host cells into which a recombinant expression vector or nucleic acid has been introduced in embodiments (1) and (2). The resulting cell or organism is a recombinant cell or organism, respectively. It is understood that such terms refer not only to the particular subject cell but also to the progeny or potential progeny of such a cell when the progeny is comprising the recombinant nucleic acid. Because certain modifications may occur in succeeding generations due to either mutation or environmental influences, such progeny may not, in fact, be identical to the parent cell, but are still included within the scope of the term as used herein, inasfar as the progeny still expresses or is able to express the recombinant protein.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e. g., linear DNA or RNA (e. g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, conjugation chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, kanamycine, tratracycleine, ampicillin and methotrexate. Nucleic acid encoding a selectable marker can be introduced into a host cell on the same vector as that encoding the above-mentioned modified nucleotide sequences or can be introduced on a separate vector. Cells stably transfected with the introduced nucleic acid can be identified by drug selection (e. g., cells that have incorporated the selectable marker gene will survive, while the other cells die).

When plasmids without an origin of replication and two different marker genes are used (e.g. pClik int sacB), it is also possible to generate marker-free strains which have part of the insert inserted into the genome. This is achieved by two consecutive events of homologous recombination (see also Becker et al., APPLIED AND ENVIRONMENTAL MICROBIOLOGY, 71 (12), p. 8587-8596; Eggeling and Bott (eds) Handbook of Corynebacterium (Taylor and Francis Group, 2005).). The sequence of plasmid pClik int sacB can be found in WO2005/059093 as SEQ ID NO:24; therein, the plasmid is called pCIS.

In another embodiment, recombinant microorganisms for use in embodiments (1) and (2) can be produced which contain selected systems which allow for regulated expression of the introduced gene. For example, inclusion of a nucleotide sequence on a vector placing it under control of the lac operon permits expression of the gene only in the presence of IPTG. Such regulatory systems are well known in the art.

Growth of Escherichia coli and Corynebacterium glutamicum-Media and Culture Conditions

In one embodiment, the method comprises culturing the microorganism in a suitable medium for methionine production. In another embodiment, the method further comprises isolating the methionine from the medium or the host cell.

The person skilled in the art is familiar with the cultivation of common microorganisms such as C. glutamicum and E. coli. Thus, a general teaching will be given below as to the cultivation of E. coli and C. glutamicum. Additional information may be retrieved from standard textbooks for cultivation of E. coli and C. glutamicum.

E. coli strains are routinely grown in MB and LB broth, respectively (Follettie et al. (1993) J. Bacteriol. 175, 4096-4103). Minimal media for E. coli is M9 and modified MCGC (Yoshihama et al. (1985) J. Bacteriol. 162,591-507), respectively. Glucose may be added at a final concentration of 1%. Antibiotics may be added in the following amounts (micrograms per millilitre): ampicillin, 50; kanamycin, 25; nalidixic acid, 25. Amino acids, vitamins, and other supplements may be added in the following amounts: methionine, 9.3 mM; arginine, 9.3 mM; histidine, 9.3 mM; thiamine, 0.05 mM. E. coli cells are routinely grown at 37 C, respectively.

Genetically modified Corynebacteria are typically cultured in synthetic or natural growth media. A number of different growth media for Corynebacteria are both well-known and readily available (Liebl et al. (1989) Appl. Microbiol. Biotechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag). Instructions can also be found in the Handbook of Corynebacterium (edited by Eggeling and Bott, ISBN 0-8493-1821-1, 2005).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, glycerol, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these 1 or (NH₄)₂SO₄, NH₄OH, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, and thiourea, sulfur containing amino acids like cysteine and other sulfur containing compounds can be used to achieve efficient methionine production. Formate may also be possible as a supplement as are other Cl sources such as methanol or formaldehyde. Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 min at 1.5 bar and 121° C.) or by sterile filtration. The components can either be sterilized together or, if necessary, separately.

All media components may be present at the beginning of growth, or they can optionally be added continuously or batchwise. Culture conditions are defined separately for each experiment.

The temperature depends on the microorgansim used and usually should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone (e.g the parent strain) or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C.

Inoculation of the media is accomplished by either introduction of a saline suspension of C. glutamicum cells from CM plates or addition of a liquid preculture of this bacterium.

Quantification of Methionine

Quantification of methionine may be performed by any textbook method known to a person skilled in the art. In the following, said quantification is exemplified.

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/1) as non-polar phase (eluent B). The following gradient is applied: Start 0% B; 39 min 39% B; 70 min 64% B; 100% B for 3.5 min; 2 min 0% B for equilibration. Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivatization is done by adding 0.5 μl of 10 mg/ml OPA reagent dissolved in 1/45/54 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. The analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) is used as internal standard

Recombination Protocol for C. glutamicum

In the following it will be described how a strain of C. glutamicum with increased efficiency of methionine production can be constructed using a specific recombination protocol.

“Campbell in,” as used herein, refers to a transformant of an original host cell in which an entire circular double stranded DNA molecule (for example a plasmid being based on pCLIK int sacB) has integrated into a chromosome by a single homologous recombination event (a cross-in event), which effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequences are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs. The “Campbell In and -Out-method” is described in WO 2007/012078 and Eggeling and Bott (eds) Handbook of Corynebacterium (Taylor and Francis Group, 2005), Chapter 23.

The present invention is described in more detail by reference to the following examples. It should be understood that these examples are for illustrative purposes only and are not to be construed as limiting the invention.

### EXAMPLES

In the following examples, standard techniques of recombinant DNA technology and molecular biology were used that were described in various publications, e.g. Sambrook et al. (2001), Molecular Cloning: A Laboratory Manual, 3rd edition, Cold Spring Harbor Laboratory Press, or Ausubel et al. (2007), Current Protocols in Molecular Biology, Current Protocols in Protein Science, edition as of 2002, Wiley Interscience. Unless otherwise indicated, all cells, reagents, devices and kits were used according to the manufacturer's instructions.

The examples of PCT/EP2007/061151 inasfar as they pertain to ICD reduction via codon usage and to its effects on production of methionine are herewith incorporated by reference. Example 1 is identical to example 3.1 of PCT/EP2007/061151.

### Example 1

### Reducing Expression of Isocitrate Dehydrogenase (icd), as Described in PCT/EP2007/061151.

Cloning

To reduce the activity of isocitrate dehydrogenase (Genbank Accession code X71489), two different changes in codon usage were made. In all cases the codons of the coding sequence were changed without changing the amino acid sequence of the encoded protein. The manipulations were all made on the only chromosomal copy of the icd gene of Corynebacterium glutamicum. The subsequent measurement of ICD activity directly allows a readout of the effect, as one can assume that it reflects the expression level given that the enzyme itself is not changed. The modifications are shown in table 1.

TABLE 1
Overview codon exchanges in ICD

| | | | affected amino acid |
|---|---|---|---|
| | name | Description | positions |
| 1 | ICD ATG → | Change of the start codon from | 1 (Met) |
| | GTG | ATG to GTG | |
| 2 | ICD CA2 | Change of a glycine and an | 32 (Gly), 33 (Ile) |
| | | isoleucine codon from GGC | |
| | | ATT to GGG ATA | |

The sequence of ICD ATG-GTG is depicted in FIG. 2a) of PCT/EP2007/061151. The sequence of ICD CA is depicted in FIG. 3a) of PCT/EP2007/061151. To introduce these mutations into the chromosomal copy of the icd coding region, 2 different plasmids were constructed which allow the marker-free manipulation by 2 consecutive homologous recombination events.

To this end the sequences of ICD ATG-GTG and ICD CA2 were cloned into the vector pClik int sacB (Becker et al (2005), Applied and Environmental Microbiology, 71 (12), p. 8587-8596) being a plasmid containing the following elements:
Kanamycin-resistance gene
SacB-gene which can be used as a positive selection marker as cells which carry this gene cannot grow on sucrose containing medium
Origin of replication for E. coli
Multiple Cloning Site (MCS)

This plasmid allows the integration of sequences at the genomic locus of C. glutamicum.

Construction of the Plasmids

All inserts were amplified by PCR using genomic DNA of ATCC 13032 as a template. The modification of the coding region was achieved by fusion PCR using the following oligonucleotides. The table shows the primers used as well as the template DNA:

TABLE 2
Overview of primers for cloning idh constructs

| | PCR A | PCR B | Fusion PCR | |
|---|---|---|---|---|
| ICD ATG → | Old 441 | Old 443 | Old 441 | Primer 1 |
| GTG | Old 444 | Old 442 | Old 442 | Primer 2 |
| | Genom. DNA | Genom. DNA | PCR A + B | Template |
| | of ATCC | of ATCC | | |
| | 13032 | 13032 | | |
| ICD CA2 | Old 441 | Old 447 | Old 441 | Primer 1 |
| | Old 448 | Old 442 | Old 442 | Primer 2 |
| | Genom. DNA | Genom. DNA | PCR A +B | Template |
| | of ATCC | of ATCC | | |
| | 13032 | ATCC 13032 | | |

Old 441 GAGTACCTCGAGCGAAGACCTCGCAGATTCCG (SEQ ID No. 6 of PCT/EP2007/061151)
Old 442 CATGAGACGCGTGGAATCTGCAGACCACTCGC (SEQ ID No. 7 of PCT/EP2007/061151)
Old 443 GAGACTCGTGGCTAAGATCATCTG (SEQ ID No. 8 of PCT/EP2007/061151)
Old 444 CAGATGATCTTAGCCACGAGTCTC (SEQ ID No. 9 of PCT/EP2007/061151)
Old 447 CTACCGCGGGGATAGAGG (SEQ ID No. 10 of PCT/EP2007/061151)
Old 448 CCTCTATCCCCGCGGTAG (SEQ ID No. 11 of PCT/EP2007/061151)

In all cases the product of the fusion PCR was purified, digested with XhoI and MluI, purified again and ligated into pClik int sacB which had been linearized with the same restriction enzymes. The integrity of the insert was confirmed by sequencing.

The coding sequence of the optimised sequence ICD ATG→GTG is shown in FIG. 2 of PCT/EP2007/061151 (SEQ ID NO:2 of PCT/EP2007/061151; SEQ ID NO:4 of present sequence listing). The coding sequence of the optimised sequence ICD CA2 is shown in FIG. 3 of PCT/EP2007/061151 (SEQ ID NO:4 of PCT/EP2007/061151; SEQ ID NO:6 of present sequence listing).

Construction of Strains with Modified ICD Expression Levels

The plasmids were then used to replace the native coding region of these genes by the coding regions with the modified coding usage. The strain used was ATCC 13032 lysC^{fbr}.

Two consecutive recombination events, one in each of the up- and the downstream region respectively, are necessary to change the complete coding sequence. The method of replacing the endogenous genes with the optimized genes is in principle described in the publication by Becker et al. (vide supra). The most important steps are:
Introduction of the plasmids in the strain by electroporation. The step is e.g. described in DE 10046870 which is incorporated by reference as far as introduction of plasmids into strains is disclosed therein.
Selection of clones that have successfully integrated the plasmid after a first homologous recombination event into the genome. This selection is achieved by growth on kanamycine-containing agar plates. In addition to that selection step, successful recombination can be checked via colony PCR. Primers used to confirm the presence of the plasmid in the genome were: BK1776 (AACGGCAGGTATATGTGATG) (SEQ ID No. 12 of PCT/EP2007/061151) and OLD 450 (CGAGTAGGTCGCGAGCAG) (SEQ ID No. 13 of PCT/EP2007/061151). The positive clones give a band of ca. 600 bp.
By incubating a positive clone in a kanamycine-free medium a second recombination event is allowed for.
Clones in which the vector backbone has been successfully removed by way of a second recombination event are identified by growth on sucrose-containing medium. Only those clones will survive that have lost the vector backbone comprising the SacB gene.
Then, clones in which the two recombination events have led to successful replacement of the native idh-coding region were identified by sequencing of a PCR-product spanning the relevant region. The PCR-product was generated using genomic DNA of individual clones as a template and primers OLD 441 and OLD 442. The PCR-product was purified and sequenced with Old 471 (GAATCCAACCCACGTTCAGGC) (SEQ ID No. 14 of PCT/EP2007/061151)

One may use different C. glutamicum strains for replacing the endogenous copy of icd. However, it is preferred to use a C. glutamicum lysine production strain such as for example ATCC13032 lysC^{fbr} or other derivatives of ATCC13032 or ATCC13286.

ATCC13032 lysC^{fbr} may be produced starting from ATCC13032. In order to generate such a lysine producing strain, an allelic exchange of the lysC wild type gene was performed in C. glutamicum ATCC13032. To this end a nucleotide exchange was introduced into the lysC gene such that the resulting protein carries an isoleucine at position 311 instead of threonine. The detailed construction of this strain is described in patent application WO2005/059093. The accession no. of the lysC gene is P26512.

To analyze the effect of the codon usage amended IDH ATG-GTG and IDH CA2, the optimized strains are compared to lysine productivity of the parent strain.

Determination of ICD Activity

One to two clones of each mutant strain were tested for ICD activity. Cells were grown in liquid culture over night at 30° C., harvested in exponential growth phase by centrifugation. The cells were washed twice with 50 mM Tris-HCl, pH 7.0. 200 mg cells were resuspended in 800 μl lysis buffer (50 mM Tris-HCl, pH 7.0, 10 mM MgCl2, 1 mM DTT, 10% Glycerol) and disrupted by bead beating (Ribolyser, 2×30 s, intensity 6). The cell debris was pelleted by centrifugation (table top centrifuge, 30 min, 13 K). The resulting supernatant is an extract of soluble proteins which was used as the following enzyme assay.

ICD activity was monitored by increase of absorption at 340 nm due to the reduction of NADP in a total volume of 1 ml under the following conditions:

30 mM Triethanolamine-chloride, pH 7.4, 0.4 mM NADP, 8 mM DL-Isocitrate, 2 mM MnSO4, cell lysate corresponding to 0.1-0.2 mg protein

ICD activities were calculated using the molar extinction coefficient of 6.22/mM*cm for NADPH.

Results

The measured ICD activities were as follows:

TABLE 3
ICD activity

| | | Specific activity of cell extract |
|---|---|---|
| Strain | Clone | U*μmol/ml*min*mg protein |
| ATCC lysC fbr | | 0.29 |
| ATCC lysC fbr | | 0.26 |
| ICD ATG → GTG | | 0.04 |
| ICD CA2 | 1 | 0.10 |
| ICD CA2 | 2 | 0.10 |

Effect on Lysine Productivity

To analyze the effect of the modified expression of ICD on lysine productivity, the optimized strains are compared to lysine productivity of the parent strains.

To this end one the strains were grown on CM-plates (10% sucrose, 10 g/l glucose, 2.5 g/l NaCl, 2 g/l urea, 10 g/l Bacto Pepton, 10 g/l yeast extract, 22 g/l agar) for 2 days at 30° C. Subsequently cells were scraped from the plates and re-suspended in saline. For the main culture 10 ml of medium I (see WO 2005/059139) and 0.5 g autoclaved CaCO₃ in a 100 ml Erlenmeyer flask were incubated together with the cell suspension up to an OD₆₀₀ of 1.5. The cells were then grown for 72 hours on a shaker of the type Infors AJ118 (Infors, Bottmingen, Switzerland) at 220 rpm.

Subsequently, the concentration of lysine that is segregated into the medium was determined. This was dome using HPLC on an Agilent 1100 Series LC system HPLC. A precolumn derivatisation with ortho-phthalaldehyde allowed to quantify the formed amino acid. The separation of the amino acid mixture can be done on a Hypersil AA-column (Agilent).

The determined lysine concentration values shown are average data from 2 independent cultivations. The deviations from the average was always below 4%.

TABLE 4
Lysine productivity

| | | Relative lysine amount | Relative OD |
|---|---|---|---|
| Strain | Clone | [%] | [%] |
| ATCC lysC fbr | | 100.00 | 100.00 |
| ATCC lysC fbr | | 99.81 | 101.22 |
| ICD ATG → GTG | | 102.34 | 92.77 |
| ICD CA2 | 1 | 101.44 | 99.80 |
| ICD CA2 | 2 | 104.85 | 96.23 |

It can be easily seen that strains with lowered ICD activity have higher lysine productivities. As all carbon source is used after 72 h, one can also directly see that the carbon yield (amount of formed product per sugar consumed) is higher in these strains.

Strain Construction for Methionine Production and Effect on Methionine Productivity

In a further experiment described in PCT/EP2007/061151, isocitrate dehydrogenase carrying the above mentioned ATG-GTG mutation in the start codon was cloned into pClik as described above leading to pClik int sacB ICD (ATG-GTG) (SEQ ID NO:15 of PCT/EP2007/061151, SEQ ID NO:5 of present sequence listing shows the vector insert). Subsequently, strain M2620 was constructed by campbelling in and campbelling out the plasmid pClik int sacB ICD (ATG-GTG) (SEQ ID NO:15 of PCT/EP2007/061151) into the genome of the strain OM469. The strain OM469 has been described in WO 2007/012078.

The strain was grown as described in WO 2007/020295. After 48 h incubation at 30° C. the samples were analyzed for sugar consumption. It was found that the strains had used up all added sugar, meaning that all strains had used the same amount of carbon source. Synthesized methionine was determined by HPLC as described above and in WO 2007/020295.

TABLE 5
Methionine production

| Strain | Methionine (mM) |
|---|---|
| OM 469 | 10.2 |
| M2620 | 23.7 |

From the data in table 5 it can be seen that the strain M2620 with an altered start codon of the ICD gene and therefore altered ICD activity has higher methionine productivity. Since all carbon source is used up after 48 h, one can also directly see, that the carbon yield (amount of formed product per sugar consumed) for the produced methionine is higher in this strain.

### Example 2

### Knock-Out of icd

To delete the icd coding region, a deletion cassette containing ˜300-600 consecutive nucleotides upstream of the icd coding sequence directly fused to 300-600 consecutive nucleotides downstream of the icd coding region is inserted into pClik int sacB. The resulting plasmid is called pClik int sacB delta icd (SEQ ID 8).

The plasmid is then transformed into C. glutamicum by standard methods, e.g. electroporation. Methods for transformation are found in e.g. Thierbach et al. (Applied Microbiology and Biotechnology 29, 356-362 (1988)), Dunican and Shivnan (Biotechnology 7, 1067-1070 (1989)), Tauch et al. (FEMS Microbiological Letters 123, 343-347 (1994)), and DE 10046870.

Two consecutive recombination events, one in each of the up- and the downstream region respectively, are necessary to delete the complete coding sequence. The method of replacing the endogenous gene with the deletion cassette using the plasmid pClik int sacB is in principle described in the publication by Becker et al. (vide supra). The most important steps are:
Selection of clones that have successfully integrated the plasmid after a first homologous recombination event into the genome. This selection is achieved by growth on kanamycine-containing agar plates. In addition to the selection step, successful recombination can be checked via colony PCR.
By incubating a positive clone in a kanamycine-free medium, a second recombination event is allowed for.
Clones in which the vector backbone has been successfully removed by way of a second recombination event are identified by growth on sucrose-containing medium. Only those clones will survive that have lost the vector backbone comprising the SacB gene.
Then, clones in which the 2 recombination events have led to the deletion of the native idh-coding region are identified with PCR-specific primers or by Southern blotting. Suitable primers are (5′ to 3′):

| | |
|---|---|
| ICD up: | GAACAGATCACAGAATCCAACC |
| ICD down: | TGGCGATGCACAATTCCTTG |

A strain in which the complete coding region of ICD was removed should result in a PCR product of about 440 base pairs (more precisely: 442 bp), while the parent strain with the wild type icd gene should show a band of about 2660 base pairs.

Successful deletion can furthermore be confirmed by Southern blotting or measuring ICD activity.

The resulting strain which contains a complete deletion of the icd coding region is called delta icd.

As this strain will lack ICD activity and therefore be unable to synthesise glutamate, it is useful to let this strain grow on rich medium or supply glutamate if grown on minimal medium.

More detailed methods on how to delete genes in C. glutamicum are also described in Eggeling and Bott (eds) Handbook of Corynebacterium” (Taylor and Francis Group, 2005) Chapter 23.8.

The effect of icd deletion on the productivity of methionine may be monitored as described above and in WO 2007/012078, WO 2007/020295.

In general, for production of methionine, the same culture medium and conditions as described in WO 2007/012078, WO 2007/020295 can be employed. The strains are precultured on CM agar overnight at 30° C. Cultured cells are harvested in a microtube containing 1.5 ml of 0.9% NaCl and cell density is determined by the absorbance at 610 nm following vortex. For the main culture, suspended cells are inoculated to reach 1.5 of initial OD into 10 ml of the production medium contained in an autoclaved 100 ml of Erlenmeyer flask having 0.5 g of CaCO3. Main culture is performed on a rotary shaker (Infors AJ118, Bottmingen, Switzerland) with 200 rpm for 48-78 hours at 30° C. For cell growth measurement, 0.1 ml of culture broth is mixed with 0.9 ml of 1 N HCl to eliminate CaCO3, and the absorbance at 610 nm is measured following appropriate dilution. The concentration of the product and residual sugar including glucose, fructose and sucrose are measured by HPLC method (Agilent 1100 Series LC system).

### Example 3

### Replacement of the Native icd Coding Region with a Variant with Lower Specific Activity

More experimental details are now described for one possible strategy to replace the original icd sequence by a mutant sequence with lower ICD activity.

1. Generation and Selection of icd Mutants with Lower Activity

In a first step, the icd coding sequence is cloned into a replicating plasmid which contains all regulatory sequences, such as promoter, RBS and a terminator sequence functioning in the host cell, which may be C. glutamicum. Ideally, a shuttle plasmid ist used which can replicate in E. coli and in C. glutamicum. An example for such a shuttle vector is pClik5aMCS (WO 2005/059093). More suitable shuttle vectors can be found in Eikmanns et al (Gene. (1991) 102, 93-8) or in the “Handbook of Corynebacterium” (edited by Eggeling and Bott, ISBN 0-8493-1821-1, 2005). One can find there a list of E. coli-C. glutamicum shuttle vectors (table 23.1) and a list of E. coli-C. glutamicum shuttle expression vectors (table 23.2). The latter are preferred as they already contain suitable promoters driving the expression of the cloned gene.

Standard methods of molecular biology, such as cloning including the amplicifation by PCR, digestion with restriction enzymes, ligation, transformation are known to the expert and can be found in standard protocol books such as Ausubel et al. (eds) Current protocols in molecular biology. (John Wiley & Sons, Inc. 2007), Sambrook et al., MOLECULAR CLONING: A LABORATORY MANUAL, Second Edition, Cold Spring Harbor Laboratory, Cold Spring Harbor, N.Y. (1989), and Ausubel et al. (eds.), SHORT PROTOCOLS IN MOLECULAR BIOLOGY, 3rd Edition (John Wiley & Sons, Inc. 1995).

A set of mutant variants of the icd coding sequence is generated by site-directed mutagenenis. Methods for mutagenesis can be found in Glick and Pasternak MOLECULAR BIOTECHNOLOGY. PRINCIPLES AND APPLICATIONS OF RECOMBINANT DNA; 2^{nd} edition (American Sicienty for Microbiology, 1998), Chapter 8: Directed Mutagenensis and Protein Engineering, and Ausubel et al. (eds) Current protocols in molecular biology. (John Wiley & Sons, Inc. 2007). Chapter 8.

The resulting set of plasmids encoding a library of icd variants is usually generated in E. coli. Subsequently, the library may be transformed into C. glutamicum by standard methods, such as electroporation. Methods for transformation are found in e.g. Thierbach et al. (Applied Microbiology and Biotechnology 29, 356-362 (1988)), Dunican and Shivnan (Biotechnology 7, 1067-1070 (1989)), Tauch et al. (FEMS Microbiological Letters 123,343-347 (1994)) or Eggeling and Bott (eds) Handbook of Corynebacterium” (Taylor and Francis Group, 2005) ISBN 0-8493-1821-1.

The resulting clones should then be tested on ICD activity. The method to measure ICD enzyme activity from crude cell extract is described in example 1.

As a control, the wild type icd gene cloned in the same plasmid as the icd variant library is determined in parallel.

Based on these results, ICD variants with lower activity compared to the wild type icd gene can be selected.

The mutants resulting in lower ICD activity can either have lower specific activity (e.g. each protein molecule is less active), be transcribed or translated less efficiently, or be less stable.

2. Replacement of the Wild Type icd Gene with a Mutant with Lower ICD Activity

To replace the wild type icd coding region by a variant with lower ICD activity, one can apply a two step strategy. In a first step, the coding region of the wild type icd gene is completely deleted from the genome. There is literature describing that cells with disrupted icd are viable. (Eikmanns et al (1995) J Bacteriol (1995) 177 (3), 774-782).

a) Deletion of Wild Type icd

The method of deletion of icd is described in example 2. The resulting strain is called delta icd.

b) Insertion of the Mutant icd Sequence

In a second step, the variant icd coding sequence is inserted into the delta icd strain. To do so, the mutant icd sequence is cloned into an suitable integration plasmid, e.g. pClik int sacB (see above) flanked by the same ˜300-600 upstream and downstream nucleotides used for the deletion construct in example 2.

Once this plasmid containing mutant icd is transformed into C. glutamicum, clones which have—after two consecutive steps of homologous recombination—inserted the mutant icd coding region into the icd locus can be identified by a similar strategy as above. PCR primers specific for the mutant ICD coding region may be used to distinguish between the delta icd strain and the positive clone.

Clones which have successfully replaced the wild type icd coding region by the mutant icd coding region will be called “icd (mut)” in the following.

3. Determination of ICD Activity

The ICD activity of strain “icd (mut)” should be compared to the activity of the parent strain containing the wild type icd gene. The method for this is described in example 1.

4. Analysis of Effects for the Production of Methionine

The above replacement of wild type icd by mutant icd may be done in different strains producing methionine by fermentation.

Suitable strains include C. glutamicum engineered to produce methionine as described in e.g. WO 2007/012078, WO 2007/020295.

The cultivation and detection for methionine production is described in the other examples. In general, for methionine, the same culture medium and conditions can be employed as described in WO 2007/012078, WO 2007/020295. The strains are precultured on CM agar overnight at 30° C. Cultured cells are harvested in a microtube containing 1.5 ml of 0.9% NaCl and cell density is determined by the absorbance at 610 nm following vortex. For the main culture, suspended cells are inoculated to reach 1.5 of initial OD into 10 ml of the production medium contained in an autoclaved 100 ml of Erlenmeyer flask having 0.5 g of CaCO3. Main culture is performed on a rotary shaker (Infors AJ118, Bottmingen, Switzerland) with 200 rpm for 48-78 hours at 30° C. For cell growth measurement, 0.1 ml of culture broth is mixed with 0.9 ml of 1 N HCl to eliminate CaCO3, and the absorbance at 610 nm is measured following appropriate dilution. The concentration of the product and residual sugar including glucose, fructose and sucrose are measured by HPLC method (Agilent 1100 Series LC system).

The accumulation of the target product methionine is expected to be higher in the strains in which ICD activity was reduced.

### Example 4

### Lowering icd Transcription/Translation by Changing the Upstream Sequence

a) Identification of a Suitable Upstream Sequence (Promoter Plus RBS)

First, an upstream sequence which is weaker than the native icd promoter has to be identified. The new upstream sequence can be derived from Corynebacterium or from other organisms. Several promoters (incl RBS) which function in bacteria, more specifically in coryneform bacteria, have been identified. Examples of such promoters are described in: DE-A-44 40 118, Reinscheid et al., Microbiology 145:503 (1999), Patek et al., Microbiology 142:1297 (1996), WO 02/40679, DE-A-103 59 594, DE-A-103 59 595, DE-A-103 59 660 and DE-A-10 2004 035 065.

In addition, other upstream regions which are weaker than the native icd promoter may be used for the replacement of the icd promoter.

The strength of upstream regions can be measured using a reporter system, such as described in Patek et al (1996) Promoters from corynebacterium glutamicum: cloning, molecular analysis and search for a consensus motif. Microbiology 142, 1297-1309.

Alternatively, one may introduce mutations in the native upstream sequence and subsequently analyze its transcriptional activity. Preferebly, the 83 nt upstream sequence of the icd start codon is used, as in this regions there is no coding region of other genes. The sequence of the upstream region is shown below (bold letters).

Methods on how to mutagenize DNA sequences including promoter sequences are well known to the expert and also described in e.g. Bernard R. Glick, Jack J. Pasternak: Molecular Biotechnology: Principles and Applications of Recombinant DNA. 2^{nd} edition. 1998. ISBN 1-55581-136-1; Chapter 8: Directed Mutagenesis and Protein engineering. A suitable promoter sequence may then be selected.

An upstream region with lower transcriptional or translational activity should be used to replace the original promoter driving ICD expression. Technically, the replacement can be done by two consecutive homologous recombination events, by the same methodology as the replacement of the icd coding region described in the previous examples.

The resulting strain will have lowered ICD activity. The effect on the productivity can be analyzed as described in Example 3.

Sequence of the ICD Gene Including 500 nt Up- and Downstream Region (SEQ ID NO:2)

Presumed promoter region (Upstream region): bold letters
bold, not underlined: (partial) 3′ coding region of the gene located upstream of icd
bold, underlined: 83 nt without any coding region

Coding region: italic

Downstream region: normal

gcgcgcatcctcgaagacctcgcagattccgatattccaggaa
ccgccatgatcgaaatcccctcagatgacgatgcacttgccat
cgagggaccttcctccatcgatgtgaaatggctgccccgcaac
ggccgcaagcacggtgaattgttgatggaaaccctggccctcc
accatgaagaaacagaagctgcagccacctccgaaggcgaact
tgtgtgggagactcctgtgttctccgccactggcgaacagatc
acagaatccaacccacgttcaggcgactactactggattgctg
gcgaaagtggtgtcgtgaccagcattcgtcgatctctagtgaa
agagaaaggcctcgaccgttcccaagtggcattcatggggtat
tggaaacacggcgtttccatgcggggctgaaactgccaccata
ggcgccagcaattagtagaacactgtattctaggtagctgaac
aaaagagcccatcaaccaaggagactcatggctaagatcatct
ggacccgcaccgacgaagcaccgctgctcgcgacctactcgct
gaagccggtcgtcgaggcatttgctgctaccgcgggcattgag
gtcgagacccgggacatttcactcgctggacgcatcctcgccc
agttcccagagcgcctcaccgaagatcagaaggtaggcaacgc
actcgcagaactcggcgagcttgctaagactcctgaagcaaac
atcattaagcttccaaacatctccgcttctgttccacagctca
aggctgctattaaggaactgcaggaccagggctacgacatccc
agaactgcctgataacgccaccaccgacgaggaaaaagacatc
ctcgcacgctacaacgctgttaagggttccgctgtgaacccag
tgctgcgtgaaggcaactctgaccgccgcgcaccaatcgctgt
caagaactttgttaagaagttcccacaccgcatgggcgagtgg
tctgcagattccaagaccaacgttgcaaccatggatgcaaacg
acttccgccacaacgagaagtccatcatcctcgacgctgctga
tgaagttcagatcaagcacatcgcagctgacggcaccgagacc
atcctcaaggacagcctcaagcttcttgaaggcgaagttctag
acggaaccgttctgtccgcaaaggcactggacgcattccttct
cgagcaggtcgctcgcgcaaaggcagaaggtatcctcttctcc
gcacacctgaaggccaccatgatgaaggtctccgacccaatca
tcttcggccacgttgtgcgcgcttacttcgcagacgttttcgc
acagtacggtgagcagctgctcgcagctggcctcaacggcgaa
aacggcctcgctgcaatcctctccggcttggagtccctggaca
acggcgaagaaatcaaggctgcattcgagaagggcttggaaga
cggcccagacctggccatggttaactccgctcgcggcatcacc
aacctgcatgtcccttccgatgtcatcgtggacgcttccatgc
cagcaatgattcgtacctccggccacatgtggaacaaagacga
ccaggagcaggacaccctggcaatcatcccagactcctcctac
gctggcgtctaccagaccgttatcgaagactgccgcaagaacg
gcgcattcgatccaaccaccatgggtaccgtccctaacgttgg
tctgatggctcagaaggctgaagagtacggctcccatgacaag
accttccgcatcgaagcagacggtgtggttcaggttgtttcct
ccaacggcgacgttctcatcgagcacgacgttgaggcaaatga
catctggcgtgcatgccaggtcaaggatgccccaatccaggat
tgggtaaagcttgctgtcacccgctcccgtctctccggaatgc
ctgcagtgttctggttggatccagagcgcgcacacgaccgcaa
cctggcttccctcgttgagaagtacctggctgaccacgacacc
gagggcctggacatccagatcctctcccctgttgaggcaaccc
agctctccatcgaccgcatccgccgtggcgaggacaccatctc
tgtcaccggtaacgttctgcgtgactacaacaccgacctcttc
ccaatcctggagctgggcacctctgcaaagatgctgtctgtcg
ttcctttgatggctggcggcggactgttcgagaccggtgctgg
tggatctgctcctaagcacgtccagcaggttcaggaagaaaac
cacctgcgttgggattccctcggtgagttcctcgcactggctg
agtccttccgccacgagctcaacaacaacggcaacaccaaggc
cggcgttctggctgacgctctggacaaggcaactgagaagctg
ctgaacgaagagaagtccccatcccgcaaggttggcgagatcg
acaaccgtggctcccacttctggctgaccaagttctgggctga
cgagctcgctgctcagaccgaggacgcagatctggctgctacc
ttcgcaccagtcgcagaagcactgaacacaggcgctgcagaca
tcgatgctgcactgctcgcagttcagggtggagcaactgacct
tggtggctactactcccctaacgaggagaagctcaccaacatc
atgcgcccagtcgcacagttcaacgagatcgttgacgcactga
agaagtaaagtctcttcacaaaaagcgctgtgcttcctcacat
ggaagcacagcgctttttcatatttttattgccataatgggca
catgcgtttttctcgagttcttcccgcacttcttatcaccacc
gccgtgagcatcccaacagcatctgctgccacactcaccgccg
acaccgacaaggaattgtgcatcgccagcaacaccgacgattc
cgcggtggttaccttctggaactccattgaagactccgtgcgc
gaacaacgcctcgacgaactagacgcccaagatccaggaatca
aagcggcgattgaaagctacatcgcccaagatgacaacgcccc
aactgctgctgaactgcaagtacgcctcgatgccatcgaatcc
ggcgaaggcctagccatgctcctcccagacgatcccacgctgg
cagaccccaacgccgaggaaagtttcaaaacggagtacacata
cgacgaagccaaagacatcatcagcggattctcca

1-6. (canceled)

7. A method for the fermentative production of methionine, comprising:
a) cultivating a microorganism in a culture medium suitable for methionine production, wherein said microorganism has been engineered to reduce its isocitrate dehydrogenase activity relative to a corresponding initial microorganism;
b) incubating said microorganism in said culture medium to allow methionine to accumulate; and
c) recovering methionine from the cells or culture medium of step b).

8. The method of claim 7, wherein the incubation of step b) lasts for a time of 48-78 hours.

9. The method of claim 7, wherein said methionine is recovered by isolating it from said culture medium.

10. The method of claim 7, wherein said microorganism is a recombinant microorganism.

11. The method of claim 10, wherein isocitrate dehydrogenase activity is reduced due to a partial or complete decrease in isocitrate dehydrogenase gene expression in said recombinant microorganism.

12. The method of claim 7, wherein said microorganism is Corynebacterium glutamicum.

13. The method of claim 7, wherein said methionine is L-methionine.

14. The method of claim 7, with the proviso that the reduction of isocitrate dehydrogenase expression in said microorganism is not due to the expression of an isocitrate dehydrogenase gene that has been modified relative to the native isocitrate dehydrogenase gene by the replacement of one or more codons with codons less frequently used by said microorganism.

15. The method of claim 11, wherein the isocitrate dehydrogenase gene in said corresponding initial microorganism encodes a protein comprising the amino acid sequence of SEQ ID NO:3.

16. The method of claim 15, wherein the isocitrate dehydrogenase gene in said recombinant organism comprises the start codon GTG.

17. The method of claim 15, wherein the isocitrate dehydrogenase gene in said recombinant organism comprises a GGG codon coding for glycine at position 32 of the isocitrate dehydrogenase enzyme and an ATA codon coding for isoleucine at position 33 of the isocitrate dehydrogenase enzyme.

18. The method of claim 11, wherein said isocitrate dehydrogenase gene in said recombinant microorganism comprises the nucleic acid sequence of SEQ ID NO:4.

19. A method for the fermentative production of methionine comprising:
a) cultivating Corynebacterium glutamicum bacteria in a culture medium suitable for methionine production, wherein said bacteria have been genetically engineered to reduce their isocitrate dehydrogenase activity relative to corresponding initial bacteria;
b) incubating said bacteria in said culture medium to allow methionine to accumulate; and
c) isolating said methionine from the culture medium of step b).

20. The method of claim 19, wherein isocitrate dehydrogenase activity in the genetically engineered bacteria is reduced due to a decrease in isocitrate dehydrogenase gene expression.

21. The method of claim 20, wherein said gene expression in said genetically engineered bacteria is reduced by at least 50% compared to expression in said corresponding initial bacteria.

22. The method of claim 21, wherein the isocitrate dehydrogenase gene in said corresponding initial bacteria encodes a protein comprising the amino acid sequence of SEQ ID NO:3.

23. The method of claim 21, wherein the isocitrate dehydrogenase gene in said genetically engineered bacteria comprises the start codon GTG.

24. The method of claim 21, wherein the isocitrate dehydrogenase gene in said genetically engineered bacteria comprises a GGG codon coding for glycine at position 32 of the isocitrate dehydrogenase enzyme and an ATA codon coding for isoleucine at position 33 of the isocitrate dehydrogenase enzyme.

25. The method of claim 21, wherein the isocitrate dehydrogenase gene in said genetically engineered bacteria comprises the nucleic acid sequence of SEQ ID NO:4.

26. The method of claim 19, wherein isocitrate dehydrogenase gene in said genetically engineered bacteria is reduced by 100% compared to expression in said corresponding initial bacteria.


SEQUENCE LISTING (9 sequences)

SEQ ID NO: 1 - DNA, 2217 bp, Corynebacterium glutamicum
  misc_feature (1)..(2217): coding sequence of isocitrate dehydrogenase
atggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 2 - DNA, 3217 bp, Corynebacterium glutamicum
  gene (1)..(3217): isocitrate dehydrogenase gene including 500 nt upstream and downstream native regions
gcgcgcatcctcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcatggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaaagtctcttcacaaaaagcgctgtgcttcctcacatggaagcacagcgctttttcatatttttattgccataatgggcacatgcgtttttctcgagttcttcccgcacttcttatcaccaccgccgtgagcatcccaacagcatctgctgccacactcaccgccgacaccgacaaggaattgtgcatcgccagcaacaccgacgattccgcggtggttaccttctggaactccattgaagactccgtgcgcgaacaacgcctcgacgaactagacgcccaagatccaggaatcaaagcggcgattgaaagctacatcgcccaagatgacaacgccccaactgctgctgaactgcaagtacgcctcgatgccatcgaatccggcgaaggcctagccatgctcctcccagacgatcccacgctggcagaccccaacgccgaggaaagtttcaaaacggagtacacatacgacgaagccaaagacatcatcagcggattctcca

SEQ ID NO: 3 - protein, 738 aa, Corynebacterium glutamicum
MAKIIWTRTDEAPLLATYSLKPVVEAFAATAGIEVETRDISLAGRILAQFPERLTEDQKVGNALAELGELAKTPEANIIKLPNISASVPQLKAAIKELQDQGYDIPELPDNATTDEEKDILARYNAVKGSAVNPVLREGNSDRRAPIAVKNFVKKFPHRMGEWSADSKTNVATMDANDFRHNEKSIILDAADEVQIKHIAADGTETILKDSLKLLEGEVLDGTVLSAKALDAFLLEQVARAKAEGILFSAHLKATMMKVSDPIIFGHVVRAYFADVFAQYGEQLLAAGLNGENGLAAILSGLESLDNGEEIKAAFEKGLEDGPDLAMVNSARGITNLHVPSDVIVDASMPAMIRTSGHMWNKDDQEQDTLAIIPDSSYAGVYQTVIEDCRKNGAFDPTTMGTVPNVGLMAQKAEEYGSHDKTFRIEADGVVQVVSSNGDVLIEHDVEANDIWRACQVKDAPIQDWVKLAVTRSRLSGMPAVFWLDPERAHDRNLASLVEKYLADHDTEGLDIQILSPVEATQLSIDRIRRGEDTISVTGNVLRDYNTDLFPILELGTSAKMLSVVPLMAGGGLFETGAGGSAPKHVQQVQEENHLRWDSLGEFLALAESFRHELNNNGNTKAGVLADALDKATEKLLNEEKSPSRKVGEIDNRGSHFWLTKFWADELAAQTEDADLAATFAPVAEALNTGAADIDAALLAVQGGATDLGGYYSPNEEKLTNIMRPVAQFNEIVDALKK

SEQ ID NO: 4 - DNA, 2217 bp, Corynebacterium glutamicum
  mutation (1)..(1): isocitrate dehydrogenase carrying an ATG-GTG mutation in the start codon
gtggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 5 - DNA, 1002 bp, Artificial Sequence
  vector insert with isocitrate dehydrogenase of Corynebacterium glutamicum carrying an ATG-GTG mutation in the start codon
ctcgagcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcgtggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccacgcgt

SEQ ID NO: 6 - DNA, 2217 bp, Corynebacterium glutamicum
  misc_feature (1)..(2217): codon usage amended isocitrate dehydrogenase (icd) CA2 carrying a mutation from GGC (Gly) ATT (Ile) into GGG ATA at amino acid positions 32 and 33
atggctaagatcatctggacccgcaccgacgaagcaccgctgctcgcgacctactcgctgaagccggtcgtcgaggcatttgctgctaccgcggggatagaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 7 - DNA, 1002 bp, Artificial Sequence
  vector insert with codon usage amended isocitrate dehydrogenase (icd) CA2 of Corynebacterium glutamicum carrying a mutation from GGC (Gly) ATT (Ile) into GGG ATA at amino acid positions 32 and 33
ctcgagcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcatggctaagatcatctggacccgcaccgacgaagcaccgctgctcgcgacctactcgctgaagccggtcgtcgaggcatttgctgctaccgcggggatagaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccacgcgt

SEQ ID NO: 8 - DNA, 5364 bp, Artificial Sequence
  plasmid pClik int sacB delta icd
tcgagaggcctgacgtcgggcccggtaccacgcgtaaaccgcagcacccgcaatcgcgcgcatcctcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcagtctcttcacaaaaagcgctgtgcttcctcacatggaagcacagcgctttttcatatttttattgccataatgggcacatgcgtttttctcgagttcttcccgcacttcttatcaccaccgccgtgagcatcccaacagcatctgctgccacactcaccgccgacaccgacaaggaattgtgcatcgccagcaacaccgacgattccgcggtggttaccttctggaactccattgaagactccgtgcgcgaacaacgcctcgacgaactagacgcccaagatccaggaatcaaagcggcgattgaaagctacatcgcccaagatgacaacgccccaactgctgctgaactgcaagtacgcctcgatgccatcgaatccggcgaaggcctagccatgctcctcccagacgatcccacgctggcagaccccaacgccgaggaaagtttcaaaacggagtacacatacgacgaagccaaagacatcatcagcggattctccagcgatccagccagcgatgtactcactagttcggacctagggatatcgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 9 - DNA, 1055 bp, Artificial Sequence
  insert of pClik int sacB delta icd
acgcgtaaaccgcagcacccgcaatcgcgcgcatcctcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcagtctcttcacaaaaagcgctgtgcttcctcacatggaagcacagcgctttttcatatttttattgccataatgggcacatgcgtttttctcgagttcttcccgcacttcttatcaccaccgccgtgagcatcccaacagcatctgctgccacactcaccgccgacaccgacaaggaattgtgcatcgccagcaacaccgacgattccgcggtggttaccttctggaactccattgaagactccgtgcgcgaacaacgcctcgacgaactagacgcccaagatccaggaatcaaagcggcgattgaaagctacatcgcccaagatgacaacgccccaactgctgctgaactgcaagtacgcctcgatgccatcgaatccggcgaaggcctagccatgctcctcccagacgatcccacgctggcagaccccaacgccgaggaaagtttcaaaacggagtacacatacgacgaagccaaagacatcatcagcggattctccagcgatccagccagcgatgtactcactagt